(19) United States
(12) Patent Application Publication
(10) Pub. No.: US 2017/0080025 A1
(43) Pub. Date: Mar. 23, 2017

(54) EFFERVESCENT GARGLE TABLET AND METHOD OF USING SAME

(71) Applicants: Timothy P. O'Connor, Scottsdale, AZ (US); Janet January Lockard-O'Connor, Scottsdale, AZ (US)

(72) Inventors: Timothy P. O'Connor, Scottsdale, AZ (US); Janet January Lockard-O'Connor, Scottsdale, AZ (US)

(21) Appl. No.: 15/370,650

(22) Filed: Dec. 6, 2016

Related U.S. Application Data
(62) Division of application No. 14/226,064, filed on Mar. 26, 2014.
(60) Provisional application No. 61/805,744, filed on Mar. 27, 2013.

(51) Int. Cl. A61K 33/14; A61K 9/46

(52) U.S. Cl. CPC A61K 33/14; A61K 9/0007

(57) ABSTRACT

An anhydrous composition is provided. The anhydrous composition may be a tablet configured to dissolve in water, the tablet including Vitamin C (as ascorbic acid), Vitamin E (as dl-alpha-tocopheryl acetate), Magnesium (as magnesium sulfate and magnesium oxide), Zinc (as zinc sulfate), Chloride (as sodium chloride), Sodium (as sodium chloride, sodium bicarbonate and Sodium carbonate), Potassium (as potassium bicarbonate), Salt (as sodium chloride), Aloe Vera, Chamomile (as matricaria recutita), Honey, and/or Stevia Leaf extract. The anhydrous composition may further comprise Citric Acid, Sorbitol, Natural flavor, Wheat Germ Oil, and/or Magnesium Oxide.

### CROSS REFERENCE TO RELATED APPLICATION

This application is a divisional of U.S. Patent Application to O'Connor et al. entitled “EFFERVESCENT GARGLE TABLET AND METHOD OF USING SAME,” Ser. No. 14/226,064 filed Mar. 26, 2014 which claims priority to U.S. Provisional Patent Application to O'Connor et al. entitled “EFFERVESCENT GARGLE TABLET AND METHOD OF USING SAME,” Ser. No. 61/805,744 filed Mar. 27, 2013, the disclosures of which are hereby incorporated entirely herein by reference.

### BACKGROUND

Technical Field

The following relates generally to effervescent tablets, and in particular to an effervescent tablet utilized for gargling.

State of the Art

Gargling is the act of washing or rinsing the throat or mouth with a liquid held in the back of the mouth, near or in the upper throat, and kept in motion by a stream of air originating from the lungs. Gargling usually requires that the head be tilted back, which provides that the mouthful of liquid can rest in the upper throat. To gargle, air is expelled from the lungs, causing the mouthful of liquid to bubble and move within the mouth and upper throat. By so doing, the mouthful of liquid can contact the mouth and throat to wash, cleanse, treat, or rinse the same.

Gargling can be effective for oral sanitation. For example, routine gargling with a mouthwash solution can help maintain a clean oral cavity and is proven to fight bad breath. A beneficial side effect may be helping to prevent the buildup of bacteria that might cause mouth, throat and upper respiratory infections. Also, gargling with medicinal antiseptic or antibiotic solutions can help to topically treat infections of the mouth and throat. Further, gargling can relieve minor mouth and throat irritations, as well as being useful in treating the symptoms of colds and the flu.

Gargling salt water may also help to alleviate the symptoms of a sore throat. The salt serves to absorb excess fluid from swollen tissues, which temporarily reduces their size and makes them less painful. The salt may also dry out any lingering bacteria in the throat. Further still, salt water may also serve to loosen up the excess mucus that coats the throat during times of sickness. The salt and the gargling action encourage the mucus to break up and dislodge from the throat.

However, the home-made remedy of gargling with warm salt water may not be appealing or even convenient. For example, too much salt in the water may cause the gargler to gag and thus dispense with gargling altogether, thus losing its potential benefits. Further, too much salt may cause the tissues to over-dehydrate causing further inflammation. On the other hand, too little salt in the water may not be effective in providing the benefits that gargling with warm salt water can provide. Moreover, the taste and/or smell of salt water may preclude many garglers from even attempting the task. And, even if the gargler is prepared to gargle, the availability of enough salt to create a warm salt water mix for gargling may not be readily accessible when needed.

Thus, there is a need in the medical and oral health industry for a tablet and a method of using the same that garglers will employ to maximize the benefits of gargling.

### SUMMARY

The following relates to effervescent tablets, and in particular to an effervescent tablet utilized for gargling.

A first general aspect relates to an effervescent tablet comprising an effervescent agent comprising an acid and a base, from 5% by weight to 40% by weight of the acid, from 10% by weight to 70% by weight of the base, and at least 15% by weight sodium chloride, wherein the tablet is configured to disintegrate in water having a temperature of between 22° and 24° C. in less than 120 seconds.

Another general aspect relates to wherein the tablet comprises at least 1000 g of sodium chloride.

Another general aspect relates to the tablet further comprising from 0.25% by weight to 20% by weight honey.

Another general aspect relates to the tablet further comprising from 0.25% by weight to 10% by weight aloe vera.

Another general aspect relates to the tablet further comprising from 0.25% by weight to 10% by weight chamomile.

Another general aspect relates to the tablet further comprising from 0.1% by weight to 5% by weight stevia leaf extract.

Another general aspect relates to the tablet further comprising from 0.5% by weight to 4% by weight ascorbic acid.

Another general aspect relates to the tablet further comprising from 2% by weight to 4.5% by weight potassium bicarbonate.

Another general aspect relates to the tablet further comprising from 0.05% by weight to 0.45% by weight Vitamin E.

Another general aspect relates to wherein the acid is citric acid and the base is sodium bicarbonate.

Another general aspect relates to wherein the acid is citric acid and the base is magnesium oxide.

Another general aspect relates to wherein the tablet dissolves in less than 60 seconds.

Another general aspect relates to wherein the sodium chloride is at least 40% by weight.

Another general aspect relates to an effervescent tablet comprising an effervescent agent comprising an acid and a base, from 10% by weight to 40% by weight of the acid, from 10% by weight to 40% by weight of the base, at least 15% by weight sodium chloride, from 0.25% by weight to 20% by weight honey, from 0.25% by weight to 10% by weight aloe vera, from 0.25% by weight to 10% by weight chamomile, from 0.1% by weight to 5% by weight stevia leaf extract, from 0.5% by weight to 4% by weight ascorbic acid, from 2% by weight to 4.5% by weight potassium bicarbonate, from 4% by weight to 18% by weight sodium bicarbonate or sodium carbonate, from 0.1% by weight to .25% by weight zinc, and from 0.05% by weight to .45% by weight Vitamin E.

Another general aspect relates to a method of treating an individual with a sore throat, the method comprising disintegrating an effervescent tablet in at least ½ cup water (about 4 oz. or 120 ml of water) to form a resulting composition, the effervescent tablet comprising an effervescent agent comprising an acid and a base, from 5% by weight to 40% by weight of the acid, from 10% by weight to 70% by weight of the base, and at least 15% by weight sodium chloride; and orally administering the resulting composition to the individual.

Another general aspect relates to wherein the orally administering the resulting composition further comprises gargling a first half of the resulting composition for about 15 seconds, expelling the first half of the resulting solution, gargling a second half of the resulting composition for about 15 seconds, and expelling the second half of the resulting solution.

The foregoing and other features, advantages, and construction of the present disclosure will be more readily apparent and fully appreciated from the following more detailed description of the particular embodiments.

### DETAILED DESCRIPTION OF EMBODIMENTS

A detailed description of the hereinafter described embodiments of the disclosed apparatus and method are presented herein by way of exemplification and not limitation. Although certain embodiments described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise.

In medical terms, a sore throat is an acute inflammation of the mucous membrane of the lower pharynx. Tonsils and the soft palate may also be inflamed. The main indication of a sore throat is pain when swallowing and, at times, a burning sensation and tightness in the throat. Secretions may be discharged from the mucous membrane or the throat may be very dry.

A sore throat can be caused by both a virus and bacterium. A sore throat is most commonly caused by viral infections such as the common cold, but can also be brought on by anything that irritates the sensitive mucous membranes at the back of the mouth and throat. The bacteria that causes the majority of throat infections such as strep throat and tonsillitis, are Group A streptococci (Group A strep). These infections can lead to other illnesses, and usually require medical attention.

A sore throat can also be related to other problems, such as allergies, acid reflux, or the environment. For example, some throats become irritated and sore from irritants in our environment or by the excessive use of the voice. Being around smoke, drinking alcoholic beverages and eating spicy foods can cause a sore throat. Straining your voice, such as when yelling at a sporting event, can also cause a sore throat.

There are a myriad of ways one can develop a sore throat. However, regardless of the way by which the sore throat is contracted, the desire to treat and alleviate the symptoms of a sore throat is almost universally appealing. The present disclosure describes an anhydrous composition that is configured to bind together in a tablet, or sachet-like, form and thereafter completely dissolve in water to thereby be gargled to treat the symptoms of a sore throat as well as provide additional health-related benefits.

Embodiments of the anhydrous composition may comprise many forms and textures. For example, the anhydrous composition may comprise a powder, particulate material, sphere, pellets, particle or granulate. As mentioned, the powder, granulate, pellets, spheres, or particles may be in effervescent form or mixtures thereof. The composition can be formulated as a powder, granulate, pellets, spheres, particles or effervescent powder, effervescent granulate, effervescent pellets, effervescent spheres, effervescent particles or mixtures. The composition, whether as a powder, granulate, pellets, spheres, particles or their mixtures, may be formulated for reconstitution with water or another suitable liquid. Further in the alternative, the composition, whether as a powder, granulate, pellets, spheres, particles or their mixtures, may be ready to use and gargled directly from a sachet or packet after dissolving in a liquid solution. Further yet in the alternative, the composition, whether as a powder, granulate, pellets, spheres, particles or their mixtures, may be pre-mixed or otherwise pre-diluted in an aqueous solution and distributed to consumers as a ready-to-use composite liquid solution.

Embodiments of the anhydrous composition may comprise the composition being an effervescent composition or tablet that may be added to an aqueous medium, such as water, prior to oral administration. Effervescent compositions use a chemical reaction between an acid and a base to create a fizzy, tingling, and/or otherwise pleasing sensation or taste in the user's mouth. For example, and not in any way limiting, the effervescent composition may have an effervescent agent comprised of an acid and a base. The effervescent agent is activated when contacted with water, e.g., when the effervescent powder or tablet is placed in water, such as a glass of water. As the powder or tablet dissolves, the water enables the acid and base to react with each other to produce carbon dioxide gas (CO₂), which imparts carbonation to the aqueous medium to create a carbonated mixture. In some cases, the effervescent agent may be a soluble acid and an alkali metal carbonate that, with the help of water, can react to make CO₂ gas.

Embodiments of the anhydrous composition may include the soluble acid being organic or mineral acid that is safe and approved for consumption and pharmaceutical and/or nutritional purposes which provides effective and rapid effervescent disintegration upon contact with water and the alkaline effervescent compound.

Embodiments of the anhydrous composition may include the acidity for the effervescent reaction being obtained from one or more of three main sources: acids, acid anhydrides, and acid salts. The food acids may be used because they are generally regarded as safe, they occur in nature, and they are ingestible. Examples of food acids may include, but are not limited to, citric acid, tartaric acid, ascorbic acid, fumaric acid, malic acid, adipic and succinic acid, acetyl salicylic acid, nicotinic acid, mixtures thereof and the like. Citric anhydride, succinic anhyride, glutamic anhydride can be used as well. Embodiments of the anhydrous composition may include the acid salt of the composition being any suitable acid salt or any mixture of suitable salts. Examples of such a suitable acid salts or mixture of suitable salts may include sodium hydrogen phosphate, disodium dihydrogen pyrophospate, acid citrate salts, aminoacid hydrochlorides, Combinations thereof are also possible. Embodiments of the composition comprise the sources of acidity in the composition being present in the composition in an amount of from 5% by weight to about 40% by weight, from about 10% by weight to about 30% by weight, or even from about 15% by weight to about 25% by weight.

Embodiments of the anhydrous composition may further comprise base components of the acid/base effervescent agent. The base component may be capable of generating a gas such as carbon dioxide, For example, the base may comprise alkaline excipients. Further in example, the alkaline component can be any suitable alkaline effervescent compound that is safe and approved for pharmaceutical and/or nutritional purposes, and is typically organic based (e.g., an alkali metal carbonate). It can provide an effective and rapid effervescent disintegration upon contact with water and the acid compound. The base may be selected from the group consisting of carbonate salts, bicarbonate salts, and mixtures thereof, such as, for example, and not in any way limiting, sodium bicarbonate, sodium carbonate, potassium bicarbonate and potassium carbonate, sodium sesquicarbonate, sodium glycine carbonate, L-lysine carbonate, arginine carbonate, calcium carbonate, magnesium carbonate, magnesium oxide, zinc carbonate, zinc oxide, amino acid carbonates, and mixtures thereof. Alkaline earth metal carbonates and bicarbonates can be used, as well as alkali metal carbonates and bicarbonates. Embodiments of the composition comprise the base in the composition being present in the composition in an amount of from 10% by weight to about 70% by weight, from about 15% by weight to about 60% by weight, or even from about 20% by weight to about 50% by weight.

Embodiments of the anhydrous composition may further comprise pharmaceutically acceptable excipients such as diluents, binders, disintegrants, color agents, flavor agents, sweeteners, flow agents, and/or lubricants, etc.

Diluents of the anhydrous composition may be inert bulking agents which are added to active pharmaceutical ingredient to make a reasonably-sized tablet. Diluents can be, but are not limited to, anhydrous lactose, lactose monohydrate, modified lactose, dibasic calcium phosphate, tribasic calcium phosphate, microcrystalline cellulose, silicified microcrystalline cellulose, powdered cellulose, maize starch, pregelatinized starch, calcium carbonate, sucrose, glucose, dextrates, dextrins, dextrose, fructose, lactitol, maltose, mannitol, sorbitol, starch, mixtures thereof and the like.

When present, such as for embodiments of the composition in tablet form, binders of the anhydrous composition may be agents used to impart cohesive qualities to the composition. Binders impart a cohesiveness to the tablet formulation that ensures that the tablet remain intact after compression as an anhydrous composition. Tablet binders used in the anhydrous composition may be those tablet binders that are commonly used for tablet preparation. Binders may include, but are not limited to, polyvinylpyrolidone, copovidone, starches such as pregelatinized. starch or plain starch, cellulose derivatives such as hydroxypropylmethyl cellulose, ethylcellulose, hydroxypropylcellulose and carboxymethylcellulose and their salts, gelatine, acacia, agar, alginic acid,carbomer, ceratonia, chitosan, dextrates,dextrin, glycerol dibehenate, guar gum, hypromellose, inulin, magnesium aluminumsilicate, maltodextrin, poloxamer, polycarbophil, polydextrose, polyethylene oxide, polymethacrylates, sodium alginate, sucrose, sorbitol, hydrogenated vegetable oil, mixtures thereof and the like. When present, the composition may comprise a binder in an amount of from 5% by weight to about 60% by weight, from about 10% by weight to about 50% by weight, or even from about 15% by weight to about 45% by weight, or even from about 20% by weight to about 40% by weight.

Disintegrants of the anhydrous composition may be, but are not limited to, modified starches, croscarmallose sodium, carboxymethylcellulose calcium, sodium starch glycolate, crospovidone, alginic acid, calcium alginate, microcrystalline cellulose, powdered cellulose, chitosan, colloidal silicon dioxide, crospovidone, guar gum, low-substituted hydroxypropylcellulose, hydroxypropyl starch, magnesium aluminium silicate, methylcellulose, polacrilin potassium, sodium alginate, starch, pregelatinized starch, mixtures thereof and the like.

Lubricants of the anhydrous composition may prevent adhesion of the tablet composition material to equipment, reduce interparticle friction, and facilitate the ejection of the compressed tablet from the die cavity. Tablet lubricants added to the anhydrous composition may be those typically used in tablet formulations. Lubricants can include, but are not limited to, magnesium stearate, calcium stearate, hydrogenated castor oil, glyceryl behenate, glyceryl monostearate, glyceryl palmitostearate, leucine, mineral oil, light mineral oil, myristic acid, palmitic acid, polyethylene glycol, potassium benzoate, sodium benzoate, sodium lauryl sulfate, sodium stearyl fumarate, stearic acid, talc, hydrogenated vegetable oil, zinc stearate, magnesium lauryl sulphate, sodium stearyl fumarate, polyethylene glycol, stearic acid, colloidal silicon dioxide or mixtures thereof and the like. When present, the composition may comprise a lubricant in an amount of from 1% by weight to about 15% by weight, from about 2% by weight to about 13% by weight, or even from about 3% by weight to about 10% by weight, or even from about 3% by weight to about 8% by weight.

Embodiments of the anhydrous composition may further comprise flavor enhancers, preservatives, antiadherants, flavorings, coloring agents, and other excipients of common use Additives ingredients may vary according to the type of compositions being manufactured.

The effervescent composition is water soluble and rapidly disintegrates. The effervescent composition may be configured to dissolve to a clear solution when placed in water at room temperature (between 22°-24° C.) in less than 1 minute, or even in less than 45 seconds. The composition preferably is self-mixing, i.e., when excess water is added to the effervescent composition, the effervescent composition will dissolve on its own without mixing or stirring from another source. The composition is palatable, can be easily swallowed, although not recommended.

The effervescent composition is also stable. While in a package, the package will exhibit minimal visible puffing, and may even remain free of visible puffing for a period of multiple days and even multiple weeks.

Embodiments of the anhydrous composition may comprise Vitamin C (as ascorbic acid), Vitamin E (as dl-alpha-tocopheryl acetate), Magnesium (as magnesium sulfate and magnesium oxide), Zinc (as zinc sulfate), Chloride (as sodium chloride), Sodium (as sodium chloride, sodium bicarbonate and Sodium carbonate), Potassium (as potassium bicarbonate), Salt (as sodium chloride), Aloe Vera, Chamomile (as matricaria recutita), Honey, and/or Stevia Leaf extract. Embodiments of the anhydrous composition may further comprise Citric Acid, Sorbitol, Natural flavor, Wheat Germ Oil, Menthol and/or Magnesium Oxide.

One characteristic feature of the disclosed anhydrous composition is that the percentage of Vitamin C (as ascorbic acid), in the total weight of the composition may be in the range of 0.5% to 4.0%, can preferably be in the range of 1.5% to 2.5%, and most preferably may be about 1.91% by weight. Ascorbic acid, the natural form of Vitamin C, is the most effective for topical administration, because it can be absorbed topically. In fact, topical Vitamin C can be a powerful source for the skin to repair itself. It is also a potent antioxidant and has the ability to fight free radicals. Antioxidants are widely used in dietary supplements and have been investigated for the prevention of diseases such as cancer, coronary heart disease and even altitude sickness which is ascorbic acid. Topical Vitamin C may be useful against inflammatory lesions and it is essential part of skin health. Gargling the composition, including the ascorbic acid, topically applies the ascorbic acid to the affected areas within the throat and mouth to provide the benefits described herein. For example, the gargled composition may help to keep the Vitamin C levels in mouth and throat tissues high.

One characteristic feature of the disclosed anhydrous composition is that the percentage of Vitamin E (as dl-alpha-tocopheryl acetate), in the total weight of the composition may be in the range of 0.05% to 0.45%, can preferably be in the range of 0.2% to 0.38%, and most preferably may be about 0.29% by weight. Vitamin E is considered to be an anti-inflammatory agent in the skin and has been shown to reduce all signs of skin inflammation. Vitamin E can be absorbed through the skin as dl-alpha-tocopheryl acetate, which allows Vitamin E to soothe irritated tissues topically. Gargling the composition, including the dl-alpha-tocopheryl acetate, topically applies the dl-alpha-tocopheryl acetate to the affected areas within the throat and mouth to provide the benefits described herein. For example, the gargled composition may help to keep the Vitamin E levels in mouth and throat tissues high.

One characteristic feature of the disclosed anhydrous composition is that the percentage of Magnesium (as magnesium sulfate and magnesium oxide), in the total weight of the composition may be in the range of 0.25% to 2.0%, can preferably be in the range of 0.5% to 1.5%, and most preferably may be about 1.06% by weight.

One characteristic feature of the disclosed anhydrous composition is that the percentage of Zinc (as zinc sulfate), in the total weight of the composition may be in the range of 0.10% to 0.25%, can preferably be in the range of 0.13% to 0.22%, and most preferably may be about 0.17% by weight. Studies have shown that zinc applied topically at the beginning of a cold may reduce the duration and severity of symptoms. Positive results have been shown in sore throats, where sore throat disappeared after 1 day in the treated group versus 3 days in the placebo group. Zinc is used topically in lozenge or nasal gel form for the treatment of colds. Further, zinc may serve to interfere with the action of viruses in the back of the throat or in the nose. Gargling the composition, including the zinc, topically applies the zinc to the affected areas within the throat and mouth to provide the benefits described herein. Moreover, certain sweeteners and flavorings used in lozenges can block zinc's antiviral action, as described above, however, dextrose, sucrose, mannitol, and sorbitol do not block such action, which is a reason why sorbitol is included in the disclosed composition.

One characteristic feature of the disclosed anhydrous composition is that the percentage of Chloride (as sodium chloride), in the total weight of the composition may be in the range of 12.0% to 20.0%, can preferably be in the range of 14.0% to 18.0%, and most preferably may be about 16.0% by weight.

One characteristic feature of the disclosed anhydrous composition is that the percentage of Sodium (as sodium chloride, sodium bicarbonate and/or Sodium carbonate), in the total weight of the composition may be in the range of 4.0% to 18.0%, can preferably be in the range of 8.0% to 15.0%, and most preferably may be about 13.4% by weight. Sodium bicarbonate, or sodium hydrogen carbonate, is the chemical compound with the formula NaHCO₃. Sodium bicarbonate is a white solid that is crystalline but often appears as a fine powder. It has a slightly salty, alkaline taste resembling that of washing soda (sodium carbonate). The natural mineral form is nahcolite. It is a component of the mineral natron and is found dissolved in many mineral springs. Sodium bicarbonate has many related names such as baking soda, bread soda, cooking soda, and bicarbonate of soda. Sodium carbonate (also known as washing soda, soda ash and soda crystals), Na₂CO₃, is a sodium salt of carbonic acid. It most commonly occurs as a crystalline heptahydrate, which readily effloresces to form a white powder, the monohydrate. Sodium carbonate is domestically well known for its everyday use as a water softener. It can be extracted from the ashes of many plants. It is synthetically produced in large quantities from salt (sodium chloride) and limestone by a method known as the Solvay process. Sodium carbonate is used as an acidity regulator, anti-caking agent, raising agent, and stabilizer.

One characteristic feature of the disclosed anhydrous composition is that the percentage of Potassium (as potassium bicarbonate), in the total weight of the composition may be in the range of 2.0% to 4.5%, can preferably be in the range of 2.5% to 3.7%, and most preferably may be about 3.13% by weight. Potassium bicarbonate (also known as potassium hydrogen carbonate or potassium acid carbonate) is a colorless, odorless, slightly basic, salty substance. Physically, potassium bicarbonate occurs as a crystal or a soft white granular powder. It may be used as a base in foods and to regulate pH. Potassium bicarbonate can be used to soften the effect of effervescence.

One characteristic feature of the disclosed anhydrous composition is that the percentage of Salt (as sodium chloride, also known as salt, common salt, table salt, or halite), in the total weight of the composition may be in the range of 10.0% to 40.0%, can preferably be in the range of 15.0% to 35.0%, and most preferably may be about 26.6% by weight. Table salt is an ionic compound with the formula NaCl, representing equal proportions of sodium and chlorine. Sodium chloride is the salt most responsible for the salinity of the ocean and of the extracellular fluid of many multicellular organisms. As the major ingredient in edible salt, it is commonly used as a condiment and food preservative. Salt is a natural disinfectant and may also improve circulation to the affected areas of the mouth and throat by coming into contact therewith by gargling. Increased blood supply provides more oxygen and faster healing, and also brings antibodies to the area more quickly and efficiently. Further, salt can draw excess fluid from inflamed tissues in the throat, drying membranes and relieving swelling and pain. Salt may also help loosen thick mucus in the throat, which thereby removes irritants like allergens, bacteria and fungi that may otherwise be present in the mucus. Gargling the composition, including the salt, topically applies the salt to the affected areas within the throat and mouth to provide the benefits described herein. The total amount of sodium chloride in the composition may be at least 15% to 25% by weight. Adding in other potential sources of sodium chloride, e.g., from the chloride and sodium described above, and the total amount of the sodium chloride in the composition may be at least 30% by weight, may be at least 40% by weight, may be at least 45% by weight, and may be at least 50% by weight and upwards of about 60% by weight.

One characteristic feature of the disclosed anhydrous composition is that the percentage of Aloe Vera, in the total weight of the composition may be in the range of 0.25% to 10%, can preferably be in the range of 0.7% to 5%, and most preferably may be about 1.06% by weight. Topical aloe gel may have immunomodulatory properties that may improve wound healing and skin inflammation. Aloe is often used to treat wounds, skin infections, burns and numerous other dermatologic conditions. Further, according to natural healing research, topical aloe significantly enhances the rate of wound healing. Gargling the composition, including the aloe vera, topically applies the aloe vera to the affected areas within the throat and mouth to provide the benefits described herein.

One characteristic feature of the disclosed anhydrous composition is that the percentage of Chamomile (as matricaria recutita), in the total weight of the composition may be in the range of 0.25% to 10%, can preferably be in the range of 0.7% to 5%, and most preferably may be about 1.06% by weight. Chamomile may have anti-inflammatory and spasmolytic effects and may promot mucosal healing. Chamomile can be made from the flowering tops of the Matricaria recutita, and can be an effective remedy for a number of various ailments. Chamomile is commonly considered the world's most soothing herb. In addition, Chamomile fights bacterial infections by destroying the very bacteria that cause the related infections. For example, one of chamomile's active ingredients, Azulene, directly fights staphylococcus and streptococcus infections. By destroying the bacteria that cause infections, chamomile helps to speed up recovery time. Externally it can be used to treat skin disorders, eye inflammation, bacterial infections, mild burns, including sunburn, rashes, and sores. Chamomile is generally regarded as safe with little or no side effects. Gargling the composition, including the Chamomile, topically applies the Chamomile to the affected areas within the throat and mouth to provide the benefits described herein.

One characteristic feature of the disclosed anhydrous composition is that the percentage of Honey, in the total weight of the composition may be in the range of 0.25% to 20%, can preferably be in the range of 0.5% to 15%, can preferably be in the range of .7% to 10%, and most preferably may be about 1.06% by weight. Honey is a topical treatment for infected wounds. It can be effective on antibiotic resistant strains of bacteria. The antibacterial properties of honey include the release of low levels of hydrogen peroxide. Honey may also have an antimicrobial action against a broad spectrum of bacteria and fungi. Honey can be a remedy for the treatment of infected wounds with no adverse effects. Further, because of honey's high osmotic effect due to the high sugar content, honey has an osmolarity sufficient to inhibit microbial growth. Moreover, honey is sweet, pleasant tasting, and has an organic aroma. Gargling the composition, including the honey, topically applies the honey to the affected areas within the throat and mouth to provide the benefits described herein. Moreover, the antibacterial activity of honey could be used for prevention and reduction of dental caries. Honey from different sources contains strains of L. acidophilus 1 that produces compounds with good antibacterial activity which may be responsible for the antibacterial properties of honey.

One characteristic feature of the disclosed anhydrous composition is that the percentage of Stevia Leaf extract, in the total weight of the composition may be in the range of 0.1% to 5%, can preferably be in the range of 0.15% to 3%, and most preferably may be about 0.29% by weight. Stevia Leaf extract is a natural mint stevia flavoring. The flavoring can make the taste of the composition and the resulting gargle solution, once the composition is dissolved, more pleasant and acceptable. The flavoring is also all natural, refreshing, cooling and aromatic.

The anhydrous composition can be provided in a variety of forms including, e.g., powder (e.g., a free flowing granulation), tablet, capsule, pellet and composite. In tablet form, the anhydrous composition can have a hardness of at least 1 kilopond (Kp), from at least about 2 Kp to about 3 Kp, from about 5 Kp to about 10 Kp, or even from about 5 Kp to about 8 Kp, as measured on a standard hardness tester.

An example of an anhydrous compound, or effervescent tablet, may comprise Vitamin C (as ascorbic acid) at a total weight of about 90 mg or 0.09 g, Vitamin E (as dl-alpha-tocopheryl acetate) at a total weight of about 30 IU or 0.0135 g (1 IU being the biological equivalent of about 0.45 mg of dl-alpha-tocopherol acetate), Magnesium (as magnesium sulfate and magnesium oxide) at a total weight of about 50 mg or 0.05 g, Zinc (as zinc sulfate) at a total weight of about 8 mg or 0.008 g, Chloride (as sodium chloride) at a total weight of about 751 mg or 0.751 g, Sodium (as sodium chloride, sodium bicarbonate and Sodium carbonate) at a total weight of about 631 mg or 0.631 g, Potassium (as potassium bicarbonate) at a total weight of about 147 mg or 0.147 g, Salt (as sodium chloride) at a total weight of about 1250 mg or 1.25 g, Aloe Vera at a total weight of about 50 mg or 0.05 g, Chamomile (as matricaria recutita) at a total weight of about 50 mg or 0.05 g, Honey at a total weight of about 50 mg or 0.05 g, Stevia Leaf extract at a total weight of about 13.5 mg or 0.0135 g, and other ingredients such as Citric Acid, Sorbitol, Natural flavor, Wheat Germ Oil, Menthol, and/or Magnesium Oxide in a combined weight of about 1.596 g or, stated another way, from about 10% to 50% by weight, from about 15% to 45% by weight, from about 20% to 40% by weight, and from about 33.96% by weight. Further, each anhydrous tablet composition may weigh 4.7 grams in total weight and may comprise the above ingredients, including pharmaceutically acceptable excipients such as diluents, binders, disintegrants, and lubricants, etc. Each anhydrous tablet composition may comprise 5.5 calories and 2.1 grams carbohydrates. The composition was then placed in ½ cup of water (about 4 oz. or 12.0 ml of water) and was observed to dissolve to a clear solution in about 45 to 60 seconds.

One characteristic feature of the disclosed anhydrous composition is that the composition may be dissolved in about ½ cup of water (about 4 oz. or 120 ml of water) or other aqueous liquid desired for gargling. By dissolving the composition in ½ cup water, which has a weight of about 118.3 grams (1 cup of water weighing about 236.6 grams), the individual ingredients within the composition, and the resulting gargle solution, can be applied topically to the affected areas within the throat and mouth at the desired, or most effective, concentrations for maximum beneficial results. By dissolving the composition in the suitable amount of water, the composition may be delivered to the affected areas in a convenient and non-invasive way. Once dissolved, the composition leaves no grainy elements to gargle that might irritate and/or hurt the throat during gargling. The gargle method also provides a convenient and therapeutic delivery of a premixed recipe of salt, vitamins and herbs.

One characteristic feature of the disclosed dissolved anhydrous composition in an aqueous solution is that the percentage of Vitamin C (as ascorbic acid), in the total weight of the combined composition and aqueous solution may be in the range of 0.1% to 0.13%, can preferably be in the range of 0.04% to 0.10%, and most preferably may be about 0.07% by weight.

One characteristic feature of the disclosed dissolved anhydrous composition in an aqueous solution is that the percentage of Vitamin E (as dl-alpha-tocopheryl acetate), in the total weight of the combined composition and aqueous solution may be in the range of 0.001% to 0.02%, can preferably be in the range of 0.005% to 0.015%, and most preferably may be about 0.01% by weight.

One characteristic feature of the disclosed dissolved anhydrous composition in an aqueous solution is that the percentage of Magnesium (as magnesium sulfate and magnesium oxide), in the total weight of the combined composition and aqueous solution may be in the range of 0.005% to 0.075%, can preferably be in the range of 0.02% to 0.06%, and most preferably may be about 0.04% by weight.

One characteristic feature of the disclosed dissolved anhydrous composition in an aqueous solution is that the percentage of Zinc (as zinc sulfate), in the total weight of the combined composition and aqueous solution may be in the range of 0.001% to 0.012%, can preferably be in the range of 0.003% to 0.009%, and most preferably may be about 0.006% by weight.

One characteristic feature of the disclosed dissolved anhydrous composition in an aqueous solution is that the percentage of Chloride (as sodium chloride), in the total weight of the combined composition and aqueous solution may be in the range of 0.1% to 1.2%, can preferably be in the range of 0.3% to 0.9%, and most preferably may be about 0.6% by weight.

One characteristic feature of the disclosed dissolved anhydrous composition in an aqueous solution is that the percentage of Sodium (as sodium chloride, sodium bicarbonate and Sodium carbonate), in the total weight of the combined composition and aqueous solution may be in the range of 0.1% to 0.9%, can preferably be in the range of 0.3% to 0.7%, and most preferably may be about 0.5% by weight.

One characteristic feature of the disclosed dissolved anhydrous composition in an aqueous solution is that the percentage of Potassium (as potassium bicarbonate), in the total weight of the combined composition and aqueous solution may be in the range of 0.04% to 0.2%, can preferably be in the range of 0.08% to 0.16%, and most preferably may be about 0.12% by weight.

One characteristic feature of the disclosed dissolved anhydrous composition in an aqueous solution is that the percentage of Salt (as sodium chloride), in the total weight of the combined composition and aqueous solution may be in the range of 0.1% to 2.0%, can preferably be in the range of 0.5% to 1.5%, and most preferably may be about 1.0% by weight.

One characteristic feature of the disclosed dissolved anhydrous composition in an aqueous solution is that the percentage of Aloe Vera, in the total weight of the combined composition and aqueous solution may be in the range of 0.005% to 0.08%, can preferably be in the range of 0.02% to 0.06%, and most preferably may be about 0.04% by weight.

One characteristic feature of the disclosed dissolved anhydrous composition in an aqueous solution is that the percentage of Chamomile (as matricaria recutita), in the total weight of the combined composition and aqueous solution may be in the range of 0.005% to 0.08%, can preferably be in the range of 0.02% to 0.06%, and most preferably may be about 0.04% by weight.

One characteristic feature of the disclosed dissolved anhydrous composition in an aqueous solution is that the percentage of Honey, in the total weight of the combined composition and aqueous solution may be in the range of 0.005% to 0.08%, can preferably be in the range of 0.02% to 0.06%, and most preferably may be about 0.04% by weight.

One characteristic feature of the disclosed dissolved anhydrous composition in an aqueous solution is that the percentage of Stevia Leaf extract, in the total weight of the combined composition and aqueous solution may be in the range of 0.001% to 0.02%, can preferably be in the range of 0.005% to 0.015%, and most preferably may be about 0.01% by weight.

Compositions of the tablet may be administered for example, by having the patient or subject gargle with an appropriate composition m a manner which results in sinus, nasal passages or contiguous membranes being directly exposed to effective amounts of the composition.

To treat a sore throat or a cold, one aspect of the present method is directed to gargling the throat and mouth with the composition dissolved in water or other liquid and repeating the process as needed, or desired. The anhydrous composition of the present invention may be sold to consumers as an anhydrous compound and thereafter dissolved in the aqueous solution by the consumer just prior to the gargling process. Also, the anhydrous composition of the present invention may be pre-dissolved in the aqueous solution and sold to consumers as a combined gargling solution for the purpose of gargling. The application of the gargling solution may be similar whether the consumer purchases the anhydrous compound and thereafter creates the gargling solution by dissolving the compound of the present disclosure in water or whether the consumer purchases the pre-dissolved or pre-mixed gargling solution having the compound pre-dissolved therein. indeed, the application time of the gargling solution to the consumer, or gargler, may be similar despite the form in which the consumer acquires the product of the present disclosure.

Embodiments of the anhydrous composition dissolved in an aqueous solution may comprise Vitamin C (as ascorbic acid) at a total weight of about 90 mg or 0.09 g, Vitamin E (as dl-alpha-tocopheryl acetate) at a total weight of about 30 IU or 0.0135 g (1 IU being the biological equivalent of about 0.45 mg of dl-alpha-tocopherol acetate), Magnesium (as magnesium sulfate and magnesium oxide) at a total weight of about 50 mg or 0.05 g, Zinc (as zinc sulfate) at a total weight of about 8 mg or 0.008 g, Chloride (as sodium chloride) at a total weight of about 751 mg or 0.751 g, Sodium (as sodium chloride, sodium bicarbonate and Sodium carbonate) at a total weight of about 631 mg or 0.631 g, Potassium (as potassium bicarbonate) at a total weight of about 147 mg or 0.147 g, Salt (as sodium chloride) at a total weight of about 1250 mg or 1.25 g, Aloe Vera at a total weight of about 50 mg or 0.05 g, Chamomile (as matricaria recutita) at a total weight of about 50 mg or 0.05 g, Honey at a total weight of about 50 mg or 0.05 g, Stevia Leaf extract at a total weight of about 13.5 mg or 0.0135 g, and other ingredients such as Citric Acid, Sorbitol, Natural flavor, Wheat Germ Oil, Menthol, and/or Magnesium Oxide at a total composite weight of about 1.596 g. Further, each anhydrous tablet composition may weigh 4.7 grams in total weight and may comprise the above ingredients, including pharmaceutically acceptable excipients such as diluents, binders, disintegrants, and lubricants, etc. Each anhydrous tablet composition may comprise 5.5 calories and 2.1 grams carbohydrates. Each anhydrous tablet may be dissolved in ½ cup of aqueous solution, such as water. The above-listed ingredients may be combined with and dissolved in ½ cup water to be administered to the gargler/consumer. In other words, the composition of the above-listed ingredients may take the form of an anhydrous compound or may alternatively take the form of a liquid solution if the composition ingredients are pre-dissolved in the liquid solution.

For example, and not by way of limitation, the application time and approximate quantities in one embodiment of a method of gargling are exemplified as follows:

A cup is filled with about ½ cup of water or other suitable liquid for dissolving the above-described anhydrous composition therein.

One tablet of the anhydrous composition is placed in the cup. The water reacts with the effervescent agent in the tablet or powder composition to completely dissolve the composition in the water. Complete dissolution of the tablet in the liquid may require between 30 seconds and 120 seconds, and may require between 45 and 90 seconds and may require approximately 60 seconds or less.

The patient or subject gargles about ¼ to ½ the volume of the gargle solution as far back in the throat as possible for about 10-20 seconds, or preferably for about 15 seconds. With the head back, the solution is gargled so that it rinses high in the throat, at the entrance of the nose, and if possible, the solution may be allowed to enter the back of the nose.

Once finished with the gargle, the gargle solution may be expelled from the mouth into an appropriate receptacle.

Another ¼ to ½ remaining volume of solution may be used to repeat the step above as many times as desired by the user for as many times as the gargle solution remains.

The purpose of gargling two or more times for 15 seconds, rather than once for 30 seconds, is that it is difficult for most people to gargle vigorously for more than 15 seconds. Also the time delay between the repetitive gargle rinses permits the composition ingredients to begin their desired effects. The time delay between repetitive gargle rinses also permits the second rinse to wash away, dislodge, or otherwise remove the undesired material in the throat treated and/or affected by the first rinse.

It is best to treat a cold or influenza at the first sign of an irritation in nose or throat. Entrenched colds and sore throats, although showing immediate relief, usually need chlorine dioxide treatments for several days. Flu and bronchitis are more difficult to treat after becoming internalized Gargling can also reduce the chances of getting an upper respiratory tract infection (URTI).

While this disclosure has been described in conjunction with the specific embodiments outlined above, it is evident that many alternatives, modifications and variations will be apparent to those skilled in the art. Accordingly, the preferred embodiments of the present disclosure as set forth above are intended to be illustrative, not limiting. Various changes may be made without departing from the spirit and scope of the present disclosure, as required by the following claims. The claims provide the scope of the coverage of the present disclosure and should not be limited to the specific examples provided herein.

What is claimed is:

1. A method of treating an individual with a sore throat, the method comprising:
disintegrating an effervescent tablet in about 120 ml of water to form a resulting composition, the effervescent tablet comprising:
an effervescent agent comprising an acid and a base;
from 5% by weight to 40% by weight of the acid;
from 10% by weight to 70% by weight of the base; and
at least 15% by weight sodium chloride; and
orally administering the resulting composition to the individual.

2. The method of claim 1, wherein the orally administering the resulting composition further comprises:
gargling a first half of the resulting composition for about 15 seconds;
expelling the first half of the resulting solution;
gargling a second half of the resulting composition for about 15 seconds; and
expelling the second half of the resulting solution.
